(19) United States
(12) Patent Application Publication
(10) Pub. No.: US 2008/0148430 A1
(43) Pub. Date: Jun. 19, 2008

(54) Peptides, Nucleic Acids and Materials

(75) Inventor: Scott Charles Peck, Columbia, MO (US)

Correspondence Address: SPECKMAN LAW GROUP PLLC, 1201 THIRD AVENUE, SUITE 330, SEATTLE, WA, 98101, US

(73) Assignee: PLANT BIOSCIENCE LIMITED, Norwich (GB)

(21) Appl. No.: 11/816,268

(22) PCT Filed: Feb. 15, 2006
(86) PCT No.: PCT/GB06/00523

§ 371 Date: Aug. 14, 2007

(30) Foreign Application Priority Data
Feb. 17, 2005 (GB) 0503342.8

(51) Int. Cl. C12N 15/82; C12Q 1/48; C12N 9/12; C07H 21/04; A01H 5/00; A01H 1/00; C12N 5/00; C12N 15/74; C12N 15/00

(52) U.S. Cl. 800/278; 435/15; 435/194; 536/23.2; 435/252.3; 435/471; 435/320.1; 435/468; 800/295; 800/317.4; 800/306; 800/317.3; 536/23.1

(57) ABSTRACT

The invention provides methods for assaying for the presence or activity of a MAP kinase in a sample, the methods comprising: (a) contacting the sample under conditions necessary for a phosphorylation reaction with a peptide substrate comprising the following target motif: HP(x)SPR, wherein: (x) represents any amino acid; (b) assessing any resulting phosphorylation of the peptide substrate; (c) optionally correlating the result from step (b) with the presence or activity of MAP kinase in the sample. Examples substrates include the Arabidopsis protein AtPhos34. In another aspect the invention provides methods and materials for influencing the salt and\or drought tolerance phenotype of a plant, for example by use of nucleic acid encoding AtPhos34 or variants thereof.

### TECHNICAL FIELD

The present invention relates generally to novel methods and substrates for measuring or detecting Mitogen-Activated Protein (MAP) kinase activity.

It further relates to methods and materials for manipulating drought or salt tolerance in plants.

### BACKGROUND ART

Research in signal transduction requires assays to monitor kinase activity.

The majority of researchers use a few general substrates for these assays. The most common of these substrates is myelin basic protein (MBP), although there are other commercially available proteins including ‘real’ substrates for mammalian kinases.

MBP is isolated from animal sources, typically brains. Therefore, it is costly to produce, and typical assay experiments using it are correspondingly costly to perform. Further, with the recognition of transmissible diseases such as scrapie, spongeform encephalitis, and the like, handling of brain tissue and materials derived therefrom has become less and less desirable.

Thus it can be seen that a new source of substrate for MAP kinases would provide a contribution to the art.

A further requirement in the art is for novel means for manipulating (generally increasing) drought or salt tolerance in plants. This is important, for example in extending the geographical range or crop or other plants, and there are numerous publications concerned with this problem.

For example WO00/11138 (Rutgers, The State University of New Jersey) concerns the use of transgenic plants expressing a betaine aldehyde dehydrogenase encoding transgene to increase salt tolerance.

Ulm et al. (2002) The EMBO Journal Vol. 21 No. 23 pp. 6483-6493, 2002 “Distinct regulation of salinity and genotoxic stress responses by Arabidopsis MAP kinase phosphatase 1” demonstrated that knockout of a certain Map Kinase regulators (MKP1) caused increased resistance to salinity.

Thus it can be seen that further means for manipulating (generally increasing) salt and\or drought tolerance in plants would provide a contribution to the art.

### DISCLOSURE OF THE INVENTION

As described in one aspect of the invention hereinafter, the present inventors have discovered that particular identified proteins from plants that are substrates for MAP kinases when overexpressed in plants give salt resistance, while knockouts are hypersensitive to salt. This is demonstrated with respect to the protein AtPhos34 in the Examples hereinafter. These plants also demonstrate drought tolerance.

Interestingly, the AtPhos34 overexpressing plants showed the salt tolerance phenotype in the adult phase of the plant, which contrasts with the MKP1 phenotype of Ulm et al. (2002)[supra], which was in the seedlings but not the adult. Additionally the MKP1 mutants were hypersensitive to UV light, whereas this was not noted for the AtPhos34 plants.

The present inventors have further identified proteins and peptides from plants that are substrates for MAP kinases and which may be produced on a large-scale in bacteria or through over-expression in plants or other recombinant systems. Thus the proteins may be used as a cheaper or more convenient alternative substrate for in vitro kinase assays to the currently more commonly used MBP.

The inventors have further characterised the precise site of phosphorylation of the proteins, thereby providing for the generation of substrate derivatives e.g. ‘super substrates’ including repeats of the target sequence.

One preferred embodiment of the invention is based on the protein AtPhos32, which expresses to extremely high levels in bacteria. Additionally the native sequence binds extremely well to nickel-nitrilotriacetic acid Ni-NTA columns. Such metal-affinity chromatography matrices are commercially available (e.g. from Qiagen) and are suitable for biomolecules which have been tagged with 6 consecutive histidine residues or which inherently bind well to the matrix under non-denaturing conditions, making a large-scale one-step purification extremely easy and cost-effective. The protein is extremely stable in 25% glycerol at −20° C. (under which conditions a single preparation has been used reliably for over 1 year) . A second protein, AtPhos34, has also been identified which serves as a substrate for MAP kinases, but this protein is less soluble and expresses to lower levels in bacteria. Several other proteins have also been identified which bear a consensus MAP-kinase phosphorylation motif (see below) and these proteins likewise are encompassed within the scope of the present invention.

Thus the present invention provides for the use of specific peptides which function as substrates for MAP kinases. The substrates include the target motif (using the single letter amino acid code):

HP(x)SPR

wherein: (x) represents any amino acid, and the underlined Ser residue (S) is phosphorylated.

Preferably the amino acid (x) is selected from S or P.

One or more sequences encoding proteins which have utility in the present invention have been published (for example from Arabidopsis—see Kerk et al (2003) Plant Physiology 131: pp 1209-1219, “Arabidopsis proteins containing similarity to the universal stress protein domain of bacteria”—Accession At5g54430). However no teaching or suggestion of the use of the sequences or proteins encoded by them in the context of the present invention has been found outside of the present disclosure.

Thus in one aspect, the invention provides a method for assaying for the presence or activity of a MAP kinase in a sample, the method comprising:

(a) contacting the sample under conditions necessary for a phosphorylation reaction with a peptide substrate comprising the following target motif:

HP(x)SPR

wherein: (x) represents any amino acid
(b) assessing any resulting phosphorylation of the peptide substrate,
(c) optionally correlating the result from step (b) with the presence or activity of MAP kinase in the sample.

MAP kinases are typically activated by the sequential actions of other protein kinases that are arranged to form a signaling cascade. where one protein kinase phosphorylates and activates the next protein kinase in sequence. Samples used in the assays can, in addition to natural MAP kinase enzymes native to the source organisms (which may be plant or animal sources), be recombinant enzymes obtained by expressing the genes encoding them using suitable expression systems. In addition, not only enzymes comprising the same amino acid sequence as natural MAP kinases, but also mutant proteins comprising only domains which support the expression of MAP kinase activity or fusion proteins in which this region is fused with another protein, can be the subject for measuring MAP kinase activity by the present invention.

Assessing the resulting phosphorylation may entail assessing (estimating or quantifying) the absolute amount of phosphorylated peptide; the ratio of the phosphorylated peptide to non-phosphorylated substrate peptide; or (where the substrate includes multiple motifs) the degree of phosphorylation of the substrate peptide molecule.

Further confirmatory steps (e.g. sequencing, use of other substrates etc.) may be performed as desired according to the use to which the assay is put.

Thus the invention also provides corresponding methods for quantifying the activity or amount of a MAP kinase in a sample.

Some further aspects and preferred embodiments of the invention will now be discussed in more detail:

### The Peptide Substrate

In the present invention, “peptide” indicates a compound comprising several amino acids bound together by a peptide bond, and is not restricted by chain length. Therefore, proteins and polypeptides are also included in “peptide” of the present invention.

Preferably the peptide substrate is at least 10, 20, 30, 40, 50, 100, 150 or 200 amino acids in length. Ideally the number of amino acids composing the peptide substrates of the present invention are kept to a minimum while still being specific for MAP kinases.

Preferably the motif is included within a contiguous sequence of at least 10, 20, 30, 40 amino acids depicted in one of SEQ ID No 2 or 3.

For example the peptide may have the structure:

R₁--(y)ₙ₁--HP(x)SPR--(y)ₙ₂--R₂,

Wherein:

R₁ is an organic molecule, a peptide, or merely an amino terminus including but not limited to H₂N;
R₂ is an organic molecule, a peptide, or merely a carboxy terminus including but not limited to —COOH and common derivatives thereof, including but not limited to an aldehyde;
(y)ₙ represents a contiguous sequence of amino acids, preferably found in SEQ ID No 2 or 3, wherein n1 is 0 or an integer of 1 to 500, preferably 1 to 16, and wherein n2 is 0 or an integer of 1 to 500, for example 2 to 238, preferably 20 to 500.

Using glutathione-S-transferase (GST) fusions, the inventors have deleted the ‘n2 region’ down to 20 amino acids and confirmed that such truncations do not affect the phosphorylation efficiency. Based on this result, it is likely that yet further truncations may be made without adversely affecting phosphorylation specificity or efficiency by MAP kinases.

Preferably n1+n2 is at least 4, 14, 24, 34 amino acids.

### Sources of Substrates

The peptide may be a naturally occurring protein or fragment thereof e.g. any of the accession sequences given in the Examples below. In one embodiment, sequence information provided herein may be used in data-base mining (e.g. of ESTs, or STSs) search to find homologous proteins including the target motif.

Alternatively substrates may be derivatives of natural sequences. Derivatives include “function-conservative variants”, which are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide.

Preferably the peptide comprises a sequence (including said motif and optionally further contiguous residues) which is overall 70, 80, 90, 95, 99% identical to all or part of SEQ ID No 2. A preferred alignment of two or more selected sequences in order to determine the “% identity” between the sequences is performed using for example, the CLUSTAL W program in MacVector version 6.5, operated with default parameters, including an open gap penalty of 10.0, an extended gap penalty of 0.1, and a BLOSUM-30 similarity matrix.

Derivatives also include fusion proteins or chimeric and tandem repeat sequences e.g. peptides including a plurality (2, 3, 4, 5 or more) of motifs, or motif containing structures, such as those discussed above.

Use of any of these substrates in a method for assaying for the presence or activity of a MAP kinase in a sample forms a further aspect of the present invention.

In one embodiment the peptide substrate of the present invention can be chemically synthesized by well known techniques. Of preference is the solid phase technique developed by R. B. Merrifield which permits the peptide to be built residue by residue from the carboxyl terminal amino acid to the amino terminal amino acid either manually or with an automated, commercially available synthesizer. Details of the solid phase technique are well known, such as set forth in B. Gutte and R. B. Merrifield, J. An. Chem. Soc., 91: 501 (1969); G. Barany and R. B. Merrifield, The Peptides, Vol. 2 (E. Gross and J. Meienhoffer eds.) Academic Press, New York (1979).

Preferably the substrates of the present invention are produced recombinantly by expression from recombinant nucleic acid.

Aspects of the invention cover the use of isolated nucleic acids including cDNA, RNA, genomic DNA and modified nucleic acids or nucleic acid analogs (e.g. peptide nucleic acid). Where a DNA sequence is specified, e.g. with reference to a figure, unless context requires otherwise the RNA equivalent, with U substituted for T where it occurs, is encompassed. Nucleic acid molecules according to the present invention may be provided isolated and/or purified from their natural environment, in substantially pure or homogeneous form, or free or substantially free of other nucleic acids of the species of origin. Where used herein, the term “isolated” encompasses all of these possibilities.

“Recombinant” refers to a nucleic acid originally formed in vitro, generally by the manipulation of a nucleic acid sequence using endonucleases, where the recombinant nucleic acid is in a form not typically found in nature.

As used herein, the terms “chimeric” and “heterologous” relative to a “gene”, or “gene construct”, “nucleic acid sequence” or “nucleic acid construct” are used interchangeably and refer to recombinant nucleic acid sequences which include a DNA coding sequence and control sequences required for expression of the coding sequence in a host cell. Such nucleic acids will have been introduced into said cells of the plant or an ancestor thereof, using genetic engineering, i.e. by human intervention. A heterologous gene may replace an endogenous equivalent gene, i.e. one which normally performs the same or a similar function, or the inserted sequence may be additional to the endogenous gene or other sequence.

The coding regions of such recombinant sequences may include modifications over the natural sequence from which they were derived e.g. to introduce or remove restriction endonuclease sites or alter codon usage. Alternatively changes to a sequence may produce a derivative by way of one or more of addition, insertion, deletion or substitution of one or more nucleotides in the nucleic acid, leading to the addition, insertion, deletion or substitution of one or more (e.g. several) amino acids in the encoded polypeptide as discussed above. Addition by extension one possibility e.g. addition of a His-Tag.

Thus in one aspect, the present invention provides a recombinant or otherwise novel nucleic acid including a nucleotide sequence encoding a substrate of the invention as discussed herein.

Nucleic acids may comprise, consist or consist essentially of the nucleotide sequence encoding a substrate of the invention.

Preferably the nucleic acid is an expression vector.

“Vector” is defined to include, inter alia, any plasmid, cosmid, phage etc. in double or single stranded linear or circular form which may or may not be self transmissible or mobilizable, and which can transform a prokaryotic or eukaryotic host either by integration into the cellular genome or exist extrachromosomally (e.g. autonomous replicating plasmid with an origin of replication).

In an expression vector the sequence encoding the substrate is under the control of, and operably linked to, an appropriate promoter or other regulatory elements for transcription in a host cell such as a microbial, e.g. bacterial, or plant cell.

By “promoter” is meant a sequence of nucleotides from which transcription may be initiated of DNA operably linked downstream (i.e. in the 3′ direction on the sense strand of double-stranded DNA). “Operably linked” means joined as part of the same nucleic acid molecule, suitably positioned and oriented for transcription to be initiated from the promoter. DNA operably linked to a promoter is “under transcriptional initiation regulation” of the promoter.

In one embodiment, the promoter is an inducible promoter. The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

Thus this aspect of the invention provides a nucleic acid construct, preferably an expression vector, comprising a promoter (optionally inducible) operably linked to a nucleotide sequence encoding a substrate of the present invention.

Generally speaking, those skilled in the art are well able to construct vectors and design protocols for recombinant gene expression. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, 1992.

In a further aspect of the invention, there is disclosed a host cell containing a recombinant (heterologous) construct according to the present invention, especially a plant or a microbial cell.

The term “heterologous” is used broadly in this aspect to indicate that the gene/sequence of nucleotides in question have been introduced into said host cell or cells using genetic engineering, i.e. by human intervention.

The host cell is preferably transformed by the construct, which is to say that the construct becomes established within the cell, altering one or more of the cell's characteristics and hence phenotype.

Thus a further aspect of the present invention provides a method of transforming a host cell involving introduction of a construct as described above into a host cell and causing or allowing recombination between the vector and the cell genome to introduce a nucleic acid according to the present invention into the genome.

The present invention also encompasses the substrates themselves and methods of making the substrates of the present invention by expression from encoding nucleic acid therefore under suitable conditions, which may be in suitable host cells.

Following expression, the product may be isolated from the expression system (e.g. microbial) and may be used in the assays described herein. Techniques for recovering substrate proteins from mixtures are well known to those skilled in the art, and isolation of the substrate from recombinant systems will present no burden to those of ordinary skill in the art. Typical protocols are set out “Protein Purification—principles and practice” Pub. Springer-Verlag, New York Inc (1982), and by Harris & Angal (1989) “Protein purification methods—a practical approach” Pub. O.U.P. UK, or references therein. See also “Protein folding” R Hermann, EPO Applied Technology Series Vol 12, European Patent Office, The Hague, Netherlands.

### Modes of Performing Assays:

Thus in one aspect, the methods of the present invention may be preceded by providing the substrate by expression from a recombinant nucleic acid.

The assay will generally be performed by exposing the sample or enzyme to the substrate in the presence of ATP under conditions necessary for the phosphorylation reaction (e.g. pH, temperature, metal ions etc.) which conditions are well known to those skilled in the art. A typical example is 50-100 mM Tris HCl (pH 7.4-7.5), 50 mM MgCl₂, 100 μM ATP, 2 mM DTT; added fresh day of assay. Typically, control experiments are also performed in which one or more assay component(s) is(are) lacking. The phosphorylation can then be assessed e.g. by use of labeled ATP.

In one embodiment the ATP may be radiolabeled (for example [γ-³²P]ATP) and the extent to which the peptide substrate is phosphorylated is determined by measuring the radioactivity of the phosphorylated peptide substrate, for example by liquid scintillation counting.

In another embodiment the invention provides for detecting a change in the phosphorylation level of the substrate peptide based on a change in reactivity of the substrate peptide with an antibody that identifies the phosphorylation state of the substrate peptide.

In further embodiments, the phosphorylation level of the substrate peptide is evaluated by others methods known to one skilled in the art (for instance, refer to, “Super-high sensitivity enzyme immunoassay method” Ishikawa E., Gakkai-Syuppan center (1993)).

### Antibodies

Antibodies to the substrates (e.g. phosphorylated substrates) discussed herein form a further aspect of the present invention. These have utility, for example, in the detection steps discussed above.

Antibodies are provided by conventional methods. For instance, polyclonal antibodies specific to a phosphorylated peptide are purified from antiserum obtained by immunizing an animal with the phosphorylated peptide, using an unphosphorylated peptide-immobilized column (non-specific antibody absorption column) or phosphorylated peptide-immobilized column (specific antibody purification column). Alternatively, hybridoma producing a specific antibody are cloned from hybridoma established from antibody producing cells of animals immunized with the phosphorylated peptide, by screening for a hybridoma producing the specific antibody. Screening is conducted by examining the reactivity of the culture supernatant to unphosphorylated and phosphorylated peptide by the ELISA method, and selecting clones with a high reactivity against the phosphorylated peptide. By culturing the hybridoma cloned, the desired monoclonal antibody is purified from the culture supernatant or ascites.

Antibodies may be modified in a number of ways. Indeed the term “antibody” should be construed as covering any specific binding substance having a binding domain with the required specificity. Thus, this term covers antibody fragments, derivatives, functional equivalents and homologues of antibodies, including any polypeptide comprising an immunoglobulin binding domain, whether natural or synthetic. Chimeric molecules comprising an immunoglobulin binding domain, or equivalent, fused to another polypeptide are therefore included. Cloning and expression of Chimeric antibodies are described in EP-A-0120694 and EP-A-0125023.

### Identification of MAP Kinase Modulators

The provision of novel MAP kinase substrates also provides novel means of identifying modulators of MAP kinase activity.

Thus the invention provides for methods for screening for a compound that modulates MAP kinase-mediated phosphorylation, comprising detecting whether there is a change in the level of MAP kinase-mediated phosphorylation of a substrate as described above, in the presence of a candidate compound, wherein an increase in the level of phosphorylation indicates that the compound agonizes MAP kinase-mediated phosphorylation, and a decrease in the level of phosphorylation indicates that the compound antagonizes MAP kinase-mediated phosphorylation.

In one embodiment, a method of this invention of screening for a compound that modulates (inhibits or promotes) the protein kinase activity of a MAP kinase may comprise the following steps of:

(a) incubating the protein kinase and the substrate peptide under conditions necessary for the phosphorylation reaction in the presence of a test compound,
(b) detecting a change in phosphorylation level of the substrate peptide e.g. based on a change in reactivity of the substrate peptide with an antibody that identifies the phosphorylation state of the substrate peptide, and,
(c) selecting a compound that decreases or increases the change in the phosphorylation level of the substrate peptide by comparing with the phosphorylation level of the substrate peptide in the absence of the test compound.

### Phosphatase Assays

The present invention provides a method for measuring not only MAP kinase activity, but also protein phosphatase activity and modulators of protein phosphatase activity towards the substrate peptide phosphorylated by a MAP kinase. The method of this invention for measuring protein phosphatase activity comprises the following steps of,

(a) contacting a sample with a substrate peptide comprising at least the phosphorylation site of the phosphorylated protein and incubating the substrate peptide and the sample under conditions necessary for the dephosphorylation reaction, and,
(b) detecting a change in the phosphorylation level of the substrate peptide e.g. based on a change in reactivity of the substrate peptide with an antibody that identifies the phosphorylation state of the substrate peptide. This is accomplished in the presence or absence of a compound whose activity as a protein phosphatase modulator is to be determined.

### Other Aspects

In yet another aspect of the present invention, the invention provides a composition comprising a MAP kinase and a substrate (e.g. phosphorylated substrate) as described herein e.g., for co-crystallization or other methods of structure-function analysis. The results of such structural studies permit rational drug design and development.

In yet another aspect of the present invention, the invention provides for use of the substrates described herein as competitive inhibitors of MAP-kinase mediated phosphorylation, for example in cellular assays.

The invention further provides a kit for use in the methods described herein comprising a peptide substrate as described herein, optionally including one or more of the following packaged compositions:

(a) labeled ATP;
(b) printed instructions for performing an assay as described herein;
(c) a MAP kinase;
(d) a protein phosphatase;
(e) antibodies as described above.
Salt and\or Drought Tolerance

In addition to providing novel MAP kinase substrates as described in the foregoing, a further aspect of the invention relates to use of certain of such substrates to modify the salt- and\or drought-tolerance phenotype of plants.

Preferably both of these things are manipulated simultaneously.

Thus in one aspect the invention provides a method for influencing, or affecting, the salt and\or drought tolerance phenotype of a plant, which method comprises the step of causing or allowing expression of a heterologous nucleic acid as described herein within the cells of the plant. Such expression will follow the earlier step of introducing the nucleic acid into a cell of the plant or an ancestor thereof.

The invention further provides use of nucleic acid, or encoded polypeptide product, as described herein, in for modifying the salt and\or drought tolerance phenotype of a plant.

As discussed hereinabove (under the heading “source of substrates”) the invention relates in various aspects to uses of isolated nucleic acids, to recombinant vectors and methods and uses relating to the same, and to host cells and methods of transforming them. Unless context demands otherwise, all of these things apply analogously to the aspects and preferred sequences discussed hereinafter.

In the present aspects the salt and\or drought tolerance of the plant is preferably increased, and the nucleic acid encodes AtPhos34 MAP kinase substrate (SEQ ID 2) or a variant thereof having salt- and\or drought-tolerance modifying activity. This is heterologously expressed, or over expressed, in the plant.

Preferably the nucleic acid includes a nucleotide sequence encoding (SEQ ID 2).

The nucleotide sequence may be that shown in SEQ ID 7 or SEQ ID 6, or be one that is degenerately equivalent thereto.

The nucleotide sequence may be a variant nucleotide sequence encoding a “function-conservative variant”, which in this aspect refers to salt- and\or drought-tolerance modifying activity (e.g. in the adult phase of the plant). Such variants may be derivatives of SEQ ID 7 or homologues. Changes may be desirable for a number of reasons. For instance they may introduce or remove restriction endonuclease sites or alter codon usage for enhanced expression in plants.

Such variant nucleotide sequences may be substantially homologous to the nucleotide sequence shown in SEQ ID 7—for example nucleic acids having such sequences may hybridise to a the complement of SEQ ID 7 under high stringency conditions.

Preferably variant nucleotide sequences will be at least 70, 75, 80, 85, 90, 95, or 99% identical to SEQ ID No 7, which may be assessed using the software described above.

One common formula for calculating the stringency conditions required to achieve hybridization between nucleic acid molecules of a specified sequence homology is (Sambrook et al., 1989 [supra]):

Tₘ=81.5° C.+16.6Log [Na+]+0.41(% G+C)−0.63(% formamide)−600/#bp in duplex

As an illustration of the above formula, using [Na+]=[0.368] and 50−% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such a sequence would be considered substantially homologous to the nucleic acid sequence of the present invention.

In addition to the methods described above, homologous variants in accordance with the present invention is obtainable by means of a method which includes:

(a) providing a preparation of nucleic acid, e.g. from plant cells.

Test nucleic acid may be provided from a cell as genomic DNA, cDNA or RNA, or a mixture of any of these, preferably as a library in a suitable vector. If genomic DNA is used the probe may be used to identify untranscribed regions of the gene (e.g. promoters etc.), such as are described hereinafter,

(b) providing a nucleic acid molecule which is derived from AtPhos34 MAP kinase substrate,
(c) contacting nucleic acid in said preparation with said nucleic acid molecule under conditions for hybridisation of said nucleic acid molecule to any said gene or homologue in said preparation, and,
(d) identifying said gene or homologue if present by its hybridisation with said nucleic acid molecule. Binding of a probe to target nucleic acid (e.g. DNA) may be measured using any of a variety of techniques at the disposal of those skilled in the art. For instance, probes may be radioactively, fluorescently or enzymatically labelled. Other methods not employing labelling of probe include amplification using PCR, RN'ase cleavage and allele specific oligonucleotide probing.

In particular hybridisation of nucleic acid molecules to a variant may be determined or identified indirectly, e.g. using a nucleic acid amplification reaction, particularly the polymerase chain reaction (PCR). PCR requires the use of two primers to specifically amplify target nucleic acid, so preferably two nucleic acid molecules based on AtPhos34 are used. Using RACE PCR, only one such primer may be needed (see “PCR protocols; A Guide to Methods and Applications”, Eds. Innis et al, Academic Press, New York, (1990)).

The identification of successful hybridisation is followed by isolation of the nucleic acid which has hybridised, which may involve one or more steps of PCR or amplification of a vector in a suitable host.

Recombinant vectors are discussed above. Particular of interest in the present context are nucleic acid constructs which operate as plant vectors. Specific procedures and vectors previously used with wide success upon plants are described by Guerineau and Mullineaux (1993) (Plant transformation and expression vectors. In: Plant Molecular Biology Labfax (Croy RRD ed) Oxford, BIOS Scientific Publishers, pp 121-148). Suitable vectors may include plant viral-derived vectors (see e.g. EP-A-194809).

Suitable promoters which operate in plants include the Cauliflower Mosaic Virus 35S (CaMV 35S). Other examples are disclosed at pg 120 of Lindsey & Jones (1989) “Plant Biotechnology in Agriculture” Pub. OU Press, Milton Keynes, UK. The promoter may be selected to include one or more sequence motifs or elements conferring developmental and/or tissue-specific regulatory control of expression. Inducible plant promoters include the ethanol induced promoter of Caddick et al (1998) Nature Biotechnology 16: 177-180.

If desired, selectable genetic markers may be included in the construct, such as those that confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate).

The present invention also provides methods comprising introduction of such a construct into a plant cell and/or induction of expression of a construct within a plant cell, by application of a suitable stimulus e.g. an effective exogenous inducer.

Thus in one aspect of the invention, there is disclosed a host plant cell containing a heterologous construct according to the present invention.

The host cell (e.g. plant cell) is preferably transformed by the construct, which is to say that the construct becomes established within the cell, altering one or more of the cell's characteristics and hence phenotype e.g. with respect to salt and\or drought tolerance.

Nucleic acid can be introduced into plant cells using any suitable technology, such as a disarmed Ti-plasmid vector carried by Agrobacterium exploiting its natural gene transfer ability (EP-A-270355, EP-A-0116718, NAR 12(22) 8711-87215 1984), particle or microprojectile bombardment (U.S. Pat. No. 5100792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser) other forms of direct DNA uptake (DE 4005152, WO 9012096, U.S. Pat. No. 4684611), liposome mediated DNA uptake (e.g. Freeman et al. Plant Cell Physiol. 29: 1353 (1984)), or the vortexing method (e.g. Kindle, PNAS U.S.A. 87: 1228 (1990d) Physical methods for the transformation of plant cells are reviewed in Oard, 1991, Biotech. Adv. 9: 1-11.

Agrobacterium transformation is widely used by those skilled in the art to transform dicotyledonous species.

The particular choice of a transformation technology will be determined by its efficiency to transform certain plant species as well as the experience and preference of the person practising the invention with a particular methodology of choice. It will be apparent to the skilled person that the particular choice of a transformation system to introduce nucleic acid into plant cells is not essential to or a limitation of the invention, nor is the choice of technique for plant regeneration.

Thus a further aspect of the present invention provides a method of transforming a plant cell involving introduction of a construct as described above into a plant cell and causing or allowing recombination between the vector and the plant cell genome to introduce a nucleic acid according to the present invention into the genome.

The invention further encompasses a host cell transformed with nucleic acid or a vector according to the present invention (e.g. comprising the AtPhos34 sequence) especially a plant cell. In the transgenic plant cell (i.e. transgenic for the nucleic acid in question) the transgene may be on an extra-genomic vector or incorporated, preferably stably, into the genome. There may be more than one heterologous nucleotide sequence per haploid genome.

As discussed above, nucleic acid heterologous to a plant cell may be non-naturally occurring in cells of that type, variety or species. Thus the heterologous nucleic acid may comprise a coding sequence of or derived from a particular type of plant cell or species or variety of plant, placed within the context of a plant cell of a different type or species or variety of plant. A further possibility is for a nucleic acid sequence to be placed within a cell in which it or a homologue is found naturally, but wherein the nucleic acid sequence is linked and/or adjacent to nucleic acid which does not occur naturally within the cell, or cells of that type or species or variety of plant, such as operably linked to one or more regulatory sequences, such as a promoter sequence, for control of expression.

Generally speaking, following transformation, a plant may be regenerated, e.g. from single cells, callus tissue or leaf discs, as is standard in the art. Almost any plant can be entirely regenerated from cells, tissues and organs of the plant. Available techniques are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

The production of stable, fertile transgenic plants in almost all economically relevant monocot plants is now routine (see e.g. Hiei et al. (1994) The Plant Journal 6, 271-282)). Microprojectile bombardment, electroporation and direct DNA uptake are preferred where Agrobacterium alone is inefficient or ineffective. Alternatively, a combination of different techniques may be employed to enhance the efficiency of the transformation process, eg bombardment with Agrobacterium coated microparticles (EP-A-486234) or microprojectile bombardment to induce wounding followed by co-cultivation with Agrobacterium (EP-A-486233). The generation of fertile transgenic plants has been achieved in the cereals rice, maize, wheat, oat, and barley (reviewed in Shimamoto, K. (1994) Current Opinion in Biotechnology 5, 158-162.; Vasil, et al. (1992) Bio/Technology 10, 667-674; Vain et al., 1995, Biotechnology Advances 13 (4): 653-671; Vasil, 1996, Nature Biotechnology 14 page 702).

Preferably AtPhos34-based constructs are transformed into tomato, nicotiana, and brassica to confer salt and\or drought tolerance to these crop species.

Plants which include a plant cell according to the invention are also provided.

In addition to the regenerated plant, the present invention embraces all of the following: a clone of such a plant, seed, selfed or hybrid progeny and descendants (e.g. F1 and F2 descendents).

The invention also provides a plant propagule from such plants, that is any part which may be used in reproduction or propagation, sexual or asexual, including cuttings, seed and so on. It also provides any part of these plants (e.g. edible fruits) which includes the plant cells or heterologous DNA described above.

In another aspect salt and\or drought tolerance may be modified by use of double stranded RNA (dsRNA) (Fire A. et al Nature, Vol 391, (1998)). dsRNA mediated silencing is gene specific and is often termed RNA interference (RNAi) (See also Fire (1999) Trends Genet. 15: 358-363, Sharp (2001) Genes Dev. 15: 485-490, Hammond et al. (2001) Nature Rev. Genes 2: 1110-1119 and Tuschl (2001) Chem. Biochem. 2: 239-245).

RNA interference is a two step process. First, dsRNA is cleaved within the cell to yield short interfering RNAs (siRNAs) of about 21-23 nt length with 5′ terminal phosphate and 3′ short overhangs (˜2 nt) The siRNAs target the corresponding mRNA sequence specifically for destruction (Zamore P. D. Nature Structural Biology, 8, 9, 746-750, (2001).

Thus in one embodiment, the invention provides double stranded RNA comprising an AtPhos34 encoding sequence, which may for example be a “long” double stranded RNA (which will be processed to siRNA, e.g., as described above). These RNA products may be synthesised in vitro, e.g., by conventional chemical synthesis methods.

RNAi may be also be efficiently induced using chemically synthesized siRNA duplexes of the same structure with 3′-overhang ends (Zamore P D et al Cell, 101, 25-33, (2000)). Synthetic siRNA duplexes have been shown to specifically suppress expression of endogenous and heterologeous genes in a wide range of mammalian cell lines (Elbashir S M. et al. Nature, 411, 494-498, (2001)).

Thus siRNA duplexes containing between 20 and 25 bps, more preferably between 21 and 23 bps, of the AtPhos34 sequence form one aspect of the invention e.g. as produced synthetically, optionally in protected form to prevent degradation. Alternatively siRNA may be produced from a vector, in vitro (for recovery and use) or in vivo.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1 provides a comparison of the phosphorylation efficiency of AtPhos32 and MBP by MAPK AtMPK3. AtMPK3 was immunoprecipitated from flagellin 22-elicited Arabidopsis suspension culture cells and used in in-vitro kinase assays with varying amounts of bacterially expressed AtPhos32 and myelin basic protein (MBP). a. Coomassie brilliant blue staining, b. Autoradiography. It can be seen that the AtPhos32 product on a microgram to microgram basis acts as a substrate for the AtMPK3 MAPK at least as a efficiently as MPB, which is used as the industry standard MAPK substrate.

FIG. 2 provides data showing that the first 46 amino acid of Phos32 are sufficient for MAPK specificity. Bacterially expressed proteins of either full length Phos32 (top panel) or a GST fusion, containing the first 46 amino acids of Phos32, were used as substrates for in vitro kinase assays using activated MAP kinases, AtMPK3 or AtMPK6. As a control for specificity, proteins mutated at the predicted MAP kinase phosphorylation site were found to not be efficient substrates for the kinases.

FIG. 3 provides data showing that overexpression of Phos34 results in plants more tolerant to treatment with 200 mM NaCl. Mature plants were watered from bottom with 200 mM NaCl for the number of day indicated. At day 12, all leaves on WT and Phos32 plants were completely bleached. All plants treated only with water showed no bleaching.

FIG. 4 provides data showing that overexpression of Phos34 results in plants more tolerant to drought conditions compared to wild-type.

### SEQUENCES

Seq. ID.1—Myelin Basic Protein (MBP) Bovine P02687

Seq. ID.2—Phos32 (At5g54430)

Seq. ID.3—Phos34 (At4g27320)

Seq. ID.4—Phos 32 nucleotide sequence

Seq. ID.5—Translation Phos32, starting from the ATG codon

Seq. ID.6—Phos 34, nucleotide sequence

Seq. ID.7—Translation of Phos34, starting at the ATG codon

### EXAMPLES

### Example 1

### Identification of MAP Kinase Substrates

While examining proteins that were differentially phosphorylated in response to microbial elicitor molecules, we identified two Arabidopsis phosphoproteins that intrinsically bind very well to Ni-NTA columns, referred to herein as AtPhos32 and AtPhos34. Upon further examination, we found by mass spectrometry that these proteins are 90% identical, see Seq. ID.2 and Seq. ID.3.

These proteins are previously unknown, but exhibit apparent structural similarity to Universal Stress Protein A (UspA) from bacteria. However, it should be noted that UspA does not have the phosphorylation sites found in these plant-derived proteins. Additionally, the function of that protein, and whether or not it even has a role in stress responses, is unknown. Finally it should also be noted that MAP kinases have not been reported to exist in bacteria.

By nanoESI-MS/MS, we identified the first phosphorylation site as a potential MAP kinase target (i.e. the canonical “pS-P” sequence was identified). The protein contains another 3 phosphorylated residues.

By in vitro kinase assays (immunoprecipitation of MAP kinase 3 or 6 from Arabidopsis and performing in-solution kinase assays with ³²P-γATP), we found that these proteins are excellent MAP kinase substrates.

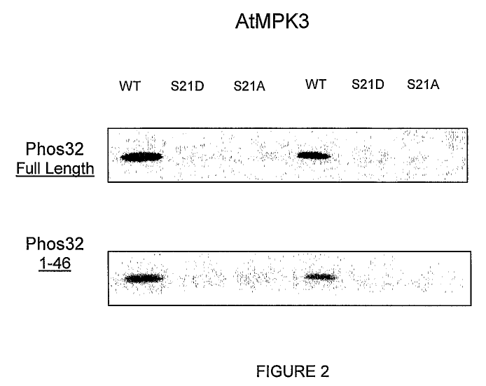

We have mutagenized the serine residue found to be phosphorylated in vivo to an alanine or aspartic acid. This mutation abolishes phosphorylation by MAP kinases in vitro. Thus, the predicted site is phosphorylated by MAP kinases (see FIG. 2).

Using this sequence information, we predicted the specificity determinants for these kinases and subsequently used this information to predict further substrates from Arabidopsis. There are 25 further proteins in Arabidopsis containing the “HPPSPR” motif found in the AtPhos32 protein, and 25 proteins with the “HPSSPR” motif found in the AtPhos34 protein (see accession numbers in Table 2). We cloned 7 of these genes into bacterial expression vectors with HIS tags to facilitate purification. We found that six out of seven bacterially expressed proteins made excellent in vitro substrates, although none had bacterial expression/isolation properties as good as those of AtPhos32 (At5g54430)—see accession numbers in Table 1:

TABLE 1
At1g15400
At1g80180
At2g14850
At3g29390
At3g16770
At1g13390

We have also performed a number of truncations of the protein, down to just the unstructured first 40 residues containing the target site with little effect on kinase phosphorylation. Thus, the sequence itself appears to be sufficient for kinase recognition. We have also tested other known kinases utilizing the AtPhos32 protein as a substrate, and found that only MAP kinases have phosphorylated this protein.

### (Predicted MAPK Substrates (PPSPR or PSSPR))

TABLE 2
At4g18880
At5g20750
At3g48530
At2g34210
At2g27210
At2g36020
At1g32120
At5g61090
At2g43680
At1g10830
At3g14067
At3g59690
At5g60470
At5g09590
At4g03080
At2g30340
At2g30615
At1g08420
At1g14690
At5g27630
At2g01190
At4g27320
At1g11360
At3g21210
At1g04360
At1g30450
At3g55980
At3g23130
At2g26530
At4g34060
At4g25770
At5g61450
At1g18410
At1g35510
At2g27350
At2g43680
At1g18620
At2g42630
At1g30810
At1g10740
At4g38430
At5g49665
At5g42390
At1g74160
At3g06170

### Example 2

### Cloning of Phos32 and Phos34

Both sequences were cloned into the NdeI site (5′ end) and BamHI site (3′ end) of the pET3a vector commercially available from Invitrogen. In both cases, the entire open reading frame of the protein was inserted with no additions or modifications to the predicted expressed protein. Constructs were transformed into standard BL21(DE3) strains of E. coli. Bacteria were grown overnight with selection, induced for 2 hours with 0.1 mM IPTG, and the cells collected by centrifugation. Proteins were isolated by sonication using either standard denaturing or native protein isolation procedures as described by company procedures for Ni-NTA resin (Qiagen).

Possibly because of an endogenous stretch of poly-histidine residues in the proteins, Phos32 and Phos34 bind to Ni-NTA with high affinity under native or denaturing conditions. Of the two, Phos32 is highly soluble in E. coli and isolates very well under native conditions with high yields. Although Phos34 can be isolated under native conditions, yields of this protein are 2-3 orders of magnitude lower.

### Example 3

### Phosphorylation of Phos32 and Pos34

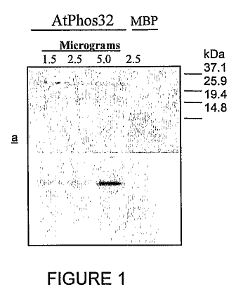

To investigate the use of these proteins as MAP kinase substrates, as compared to MBP, we used 2 μg of MBP in an in vitro kinase assay with AtMPK3 and compared the level of phosphorylation to that on 0.2 μg, 2 μg, and 10 μg of Phos32. See FIG. 1. As can be seen, Phos32 is at least as good if not a better substrate than MBP for this MAP Kinase.

### Example 4

### Truncation of the Phos32 protein

As shown in FIG. 2, the first 46 amino acids of Phos32 protein are sufficient for MAP kinase recognition and phosphorylation. FIG. 2 also demonstrates that the predicted serine residue (S21) is the MAP kinase target as mutation of this residue to alanine or aspartic acid abolishes phosphorylation by MAPKs.

### Example 5

### Antibody Data Relating to Differential Recognition of the Phosphorylated and Unphosphorylated Proteins

To detect the intact protein, we raised a polyclonal antibody raised against the bacterially expressed protein (the antibody was raised to Phos32, but recognized both proteins). To detect the phosphorylated form of the protein, we use a commercial antibody from Cell Signalling Technologies that recognizes phosphoThreonine-Proline, the phosphorylation site target of MAP kinases and cyclin dependent protein kinases. According to the manufacturer, this antibody also recognized phosphoSerine-Proline. We have used this antibody to detect differential phosphorylation of Phos32 and 34 from intact plants treated with flagellin 22.

### Example 6

### Preparation of Plants Overexpressing these Proteins which Themselves may Act as a Source for the Proteins, as an Alternate to Bacterial Expression

To make the overexpressing plants, the entire cDNA was isolated from its pSPORT vector (vector in which the Michigan State University Arabidopsis EST clone library was made) using SmaI and XbaI. This fragment was then cloned into a modified pCAMBIA2300 vector containing a 35S promoter and terminator flanking a polylinker region (this is a vector modified from the pCAMBIA collection). The constructs were then transformed into Agrobacterium by standard procedures. Arabidopsis was transformed by standard Agro dipping method. Plants were selected on Kanamycin and screened for overexpression by immunoblot analysis using the polyclonal antibody against Phos32.

### Example 7

### Salt Tolerance in Plants Overexpressing the Phos34 Protein

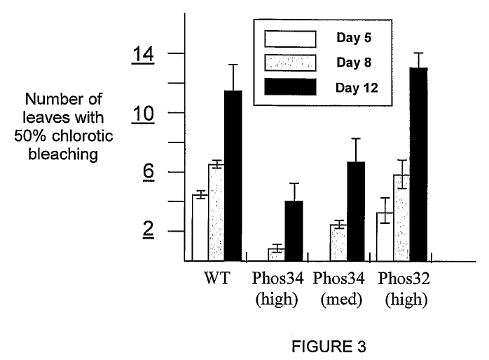

This example shows that plants overexpressing Phos34 are more tolerant to treatment with 200 mM NaCl. Six to eight week-old plants are watered from the bottom (root feeding) with the salt solution, replacing the solution when the tray is empty. Comparisons are always made between plants randomly distributed within the same tray, thus receiving the same watering treatments. Within 4-6 days after starting the treatment, leaves of wild-type (WT) plants or plants overexpressing Phos32 begin bleaching, whereas leaves of Phos34 overexpressing (OE) plants are unaffected (see FIG. 3). As the treatment continues, Phos34 OE plants remain far more tolerant, maintaining at least 50% green material even when other plants in the treatment have completely bleached and died. These effects are dose-dependent, as an independent line of Phos34 OE plants (called medium expressor) shows less—but still obvious—resistance.

Phos32 OE plants expressing to levels similar to the Phos34 OE (high) plants show no enhanced resistance, indicating that the effects are specific for the Phos34 protein. These experiments have been repeated four times with similar results.

### Example 8

### Drought Tolerance in Plants Overexpressing the Phos34 Protein

Plants were prepared as describe above.

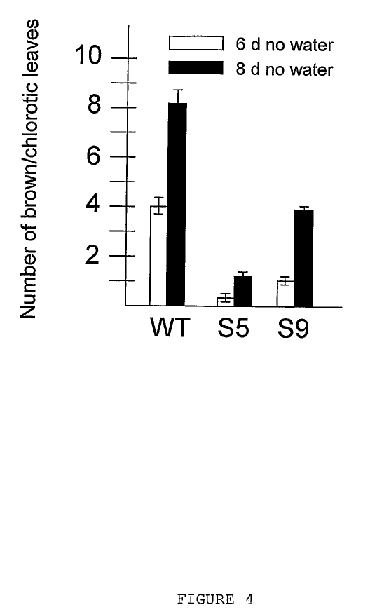

In FIG. 4, plant S5 is a ‘high’ overexpressor of Phos34 and S9 is a ‘medium’ overexpressor. The Figure shows a graph of the drought tolerance data for these plants compared to wild-type (WT).

After growing plants to maturity, water was drained from the tray containing all the plants. The plants were then left in the growth chamber without subsequent watering (pots of plants were scattered randomly in the trays to avoid positional effects). Photographs were taken 4 days after removing water (all plants still green), 6 days, and 8 days. The graph is an average of 4 plants showing the number of leaves per plant with severe browning/chlorosis.

As can be seen the drought-tolerance phenotype shows a dose-dependence on the level of protein.

```
SEQUENCES
SEQ. ID 1 - MYELIN BASIC PROTEIN (MBP), BOVINE P02687 (EMPIRICAL MAPK
SITE SHOWN)
    1 aaqkrpsqrs kylasastmd harhgflprh rdtgildslg rffgsdrgap krgsgkdghh
  61 aartthygsl pqkaqghrpq denpvvhffk nivtprtppp sqgkgrglsl srfswgaegq
121 kpgfgyggra sdyksahkgl kghdaqgtls kifklggrds rsgspmarr
SEQ. ID. 2 PHOS32 (AT5G54430)
MNPADSDHPQLPNIKIHHPPSPRHSHHHHSSSTPSSAATPTPTAGARRKIGVAVDLSEESSFAVRWAVD
HYIRPGDAVVLLHVSPTSVLFGADWGPLPLKTQIEDPNAQPQPSQEDFDAFTSTKVADLAKPLKELGFP
YKIHIVKDHDMRERLCLEIERLGLSAVIMGSRGFGAEKKRGSDGKLGSVSDYCVHHCVCPVVVVRYPDD
RDGPVPIVTVKSGGDDDGDVVAASASAHHEHIKDE
SEQ. ID. 3 PHOS34 (AT4G27320)
MNPDSDYPHLPNIKIHHPSSPRHSHHHSSSTPSAATPTPTAGARRKIGVAVDLSEESAFAVRWAVDHYI
RPGDAVVILHVSPTSVLFGADWGPLPLQTPPPPSAATDPGAQPKPSQEDFDAFTSSKVADLAKPLKEAG
FPHKIHIVKDHDMRERLCLETERLNLSAVIMGSRGFGAEKRGSDGKLGSVSDYCVHHCVCPVVVVRYPD
DRDGPAPPGNVGATREAIVTVKSRRDDDDDDDEDHEAKIAAAASDHHEHIKDE
SEQ. ID.4, PHOS32:
GAACAAAAACAACATCTGAAAAAAATC [symbol] AATCCAGCAGATTCCGATCATCCACAGCTTCCAAACATC
AAGATCCATCACCCTCCATCTCCACGTCACTCTCACCATCACCACTCCTCATCTACTCCCTCCTCCGCC
GCAACTCCAACACCAACCGCCGGAGCTCGTCGTAAAATCGGAGTCGCAGTTGATCTCTCCGAAGAAAGC
TCTTTCGCCGTTCGTTGGGCTGTAGATCACTACATCCGTCCCGGAGACGCCGTTGTTCTTCTCCACGTT
TCTCCAACCTCCGTCCTCTTCGGTGCCGATTGGGGACCTCTCCCTCTGAAAACTCAAATTGAAGATCCA
AACGCTCAACCTCAACCTAGTCAAGAGGATTTTGATGCTTTTACTTCAACAAAAGTTGCGCATCTAGCT
AAACCGTTGAAGGAGTTAGGGTTTCCTTATAAGATCCATATAGTGAAAGATCATGATATGAGAGAGAGA
TTATGTCTTGAGATTGAGAGGCTTGGTCTTAGTGCTGTGATTATGGGAAGTAGAGGTTTTGCTGCTGAG
AAAAAAAGAGGAAGTGATGGCAAGCTTGGCTCTGTTAGTGATTACTGTGTTCATCACTGTGTTTGTCCT
GTTGTTGTGGTTAGATATCCTGATGATCGTGATGGACCTGTGCCTATTGTTACTGTCAAGTCTGGTGGA
GATGATGATGGAGATGTTGTTGCTGCTTCTGCTTCTGCTCATCATGAACACATCAAAGATGAGTGAGGA
TCTAAGTAATATATCACCCTCTTCTCTTCTAGGTACACAAAGCAAGTAGGGGTGTTAGCGGAATGTTCA
GAGGAGTGTTGAATGTGTGTTCTTTCTTTAGCTATGACAATGTTGATGAATTTCTTTGGGTTATGTAAT
ATGAATGAGTGTTGAGTGGTTTCTGCATTAAACATGAAGGAGTTGTATGATTAATGAACCCCTTTTAGG
AAGAAGAAGGATTATACGTTTCTTTAATAAAGACTGGTATTTTAAAAAAAAAAAAAAAAA
SEQ. ID. 5., TRANSLATION PHOS32), STARTING FROM THE ATG CODON:
M N P A D S D H P Q L P N I K I H H P P S P R H S H H H H S S S T P
ATGAATCCAGCAGATTCCGATCATCCACAGCTTCCAAACATCAAGATCCATCACCCTCCATCTCCACGTCACTCTCACCATCACCACTCCTCATCTACTC
                  \hat{ }10 \hat{ }20 \hat{ }30 \hat{ }40 \hat{ }50 \hat{ }60 \hat{ }70 \hat{ }80 \hat{ }90
    S S A A T P T P T A G A R R K I G V A V D L S E E S S F A V R W A
CCTCCTCCGCCGCAACTCCAACACCAACCGCCGGAGCTCGTCGTAAAATCGGAGTCGCAGTTGATCTCTCCGAAGAAAGCTCTTTCGCCGTTCGTTGGGC
                  \hat{ }110 \hat{ }120 \hat{ }130 \hat{ }140 \hat{ }150 \hat{ }160 \hat{ }170 \hat{ }180 \hat{ }190
  V D H Y I R P G D A V V L L H V S P T S V L F G A D W G P L P L K
TGTAGATCACTACATCCGTCCCGGAGACGCCGTTGTTCTTCTCCACGTTTCTCCAACCTCCGTCCTCTTCGGTGCCGATTGGGGACCTCTCCCTCTGAAA
                  \hat{ }210 \hat{ }220 \hat{ }230 \hat{ }240 \hat{ }250 \hat{ }260 \hat{ }270 \hat{ }280 \hat{ }290
T Q I E D P N A Q P Q P S Q E D F D A F T S T K V A D L A K P L K E
ACTCAAATTGAAGATCCAAACGCTCAACCTCAACCTAGTCAAGAGGATTTTGATGCTTTTACTTCAACAAAAGTTGCGGATCTAGCTAAACCGTTGAAGG
                  \hat{ }310 \hat{ }320 \hat{ }330 \hat{ }340 \hat{ }350 \hat{ }360 \hat{ }370 \hat{ }380 \hat{ }390
    L G F P Y K I H I V K D H D M R E R L C L E I E R L G L S A V I M
AGTTAGGGTTTCCTTATAAGATCCATATAGTGAAAGATCATGATATGAGAGAGAGATTATGTCTTGAGATTGAGAGGCTTGGTCTTAGTGCTGTGATTAT
                  \hat{ }410 \hat{ }420 \hat{ }430 \hat{ }440 \hat{ }450 \hat{ }460 \hat{ }470 \hat{ }480 \hat{ }490
  G S R G F G A E K K R G S D G K L G S V S D Y C V H H C V C P V V
GGGAAGTAGAGGTTTTGGTGCTGAGAAAAAAAGAGGAAGTGATGGCAAGCTTGGCTCTGTTAGTGATTACTGTGTTCATCACTGTGTTTGTCCTGTTGTT
                  \hat{ }510 \hat{ }520 \hat{ }530 \hat{ }540 \hat{ }550 \hat{ }560 \hat{ }570 \hat{ }580 \hat{ }590
V V R Y P D D R D G P V P I V T V K S G G D D D G D V V A A S A S A
GTGGTTAGATATCCTGATGATCGTGATGGACCTGTGCCTATTGTTACTGTCAAGTCTGGTGGAGATGATGATGGAGATGTTGTTGCTGCTTCTGCTTCTG
                  \hat{ }610 \hat{ }620 \hat{ }630 \hat{ }640 \hat{ }650 \hat{ }660 \hat{ }670 \hat{ }680 \hat{ }690
    H H E H I K D E
CTCATCATGAACACATCAAAGATGAGTGAGGATCTAAGTAATATATCACCCTCTTCTCTTCTAGGTACACAAAGCAAGTAGGGGTGTTAGCGGAATGTTC
                  \hat{ }710 \hat{ }720 \hat{ }730 \hat{ }740 \hat{ }750 \hat{ }760 \hat{ }770 \hat{ }780 \hat{ }790
AGAGGAGTGTTGAATGTGTGTTCTTTCTTTAGCTATGACAATGTTGATGAATTTCTTTGGGTTATGTAATATGAATGAGTGTTGAGTGGTTTCTGCATTA
                  \hat{ }810 \hat{ }620 \hat{ }830 \hat{ }840 \hat{ }850 \hat{ }860 \hat{ }870 \hat{ }880 \hat{ }890
AACATGAAGGAGTTGTATGATTAATGAACCCCTTTTAGGAAGAAGAAGGATTATACGTTTCTTTAATAAAGACTGGTATTTTGAAAAAAAAAAAAAAAA
                  \hat{ }910 \hat{ }920 \hat{ }930 \hat{ }940 \hat{ }950 \hat{ }960 \hat{ }970 \hat{ }980 \hat{ }990
SEQ. ID. 6, PHOS34, FULL SEQUENCE:
AAATTGTTGTTTTATGGTGCGTCTGCGGTACGGGTGCGGAACGATC [symbol] AATCCAGATTCCGATTATCC
TCATCTCCCTAACATCAAGATCCACCATCCTTCATCTCCTCGTCACTCTCACCACCACTCTTCCTCCAC
TCCTTCCGCCGCTACTCCTACCCCAACCGCCGGTGCTCGCCGTAAGATCGGAGTCGCCCTTGACCTTTC
CGAAGAAAGCGCTTTCGCTGTTCGCTGGGCTGTCGATCATTACATCCGTCCCCGAGACGCCGTCGTCAT
TCTCCACGTTTCTCCAACCTCCGTTCTCTTCGGCGCCGATTGGGGACCTCTCCCTCTCCAAACTCCTCC
TCCTCCTTCCGCCGCTACCGATCCCGGAGCTCAGCCGAAGCCTAGTCAGGAAGATTTCGATGCGTTTAC
TTCTTCCAAAGTAGCGGATCTAGCGAAGCCGTTGAAGGAAGCTGGGTTTCCTCATAAGATCCATATAGT
GAAAGATCACGATATGAGAGAGAGGCTTTGCTTAGAGACTGAAAGGCTTAATCTAAGCGCCGTGATAAT
GGGAAGCAGAGGATTTGGTGCTGAGAAGAGAGGAAGTGATGGCAAGCTTGGCTCTGTTAGTGATTATTG
TGTTCACCATTGTGTTTGTCCTGTTGTTGTTGTTAGATATCCTGATGATCGTGATGGTCCTGCTCCTCC
TGGGAATGTTGGAGCTACCAGGGAAGCTATTGTCACTGTTAAATCACGTAGGGATGATGACGATGATGA
TGATGAAGATCATGAGGCTAAGATTGCTGCTGCTGCTTCCGATCATCATGAACACATCAAAGATGAGTA
AAGTGCCTTAGTATCTCATCAGGTTCACCCTCTTCACAGTCAGGTACAACGATGGATGAAACAAAACGG
ATTTGTGCAGAGGAGTGTGTTTGTGTGTTTGTTTATTGCTATCTTTTGCAATATTGAAAAGTCTCTGGA
ATCTCCTCTGGATTATTTATTCCCAGTCCTTGTTCTTCTTATAATTCTCCATGTATGATTTTCACATTT
AAACAATGCCCTTTTCCATGTATGACTTTCACATTTAAACAATACCCTTTTGGGGTTATGACCTTAATC
TTCAATCTAATTTGATATCAAAAAAAAAAAAAAA
SEQ. ID. 7, TRANSLATION OF PHOS34, STARTING FROM THE ATG CODON:
M N P D S D Y P H L P N I K I H H P S S P R H S H H H S S S T P S A
ATGAATCCAGATTCCGATTATCCTCATCTCCCTAACATCAAGATCCACCATCCTTCATCTCCTCGTCACTCTCACCACCACTCTTCCTCCACTCCTTCCG
                  \hat{ }10 \hat{ }20 \hat{ }30 \hat{ }40 \hat{ }50 \hat{ }60 \hat{ }70 \hat{ }60 \hat{ }90
    A T P T P T A G A R R K I G V A V D L S E E S A F A V R W A V D H
CCGCTACTCCTACCCCAACCGCCGGTGCTCGCCGTAAGATCGGAGTCGCCGTTGACCTTTCCGAAGAAAGCGCTTTCGCTGTTCGCTGGGCTGTCGATCA
                  \hat{ }110 \hat{ }120 \hat{ }130 \hat{ }140 \hat{ }150 \hat{ }160 \hat{ }170 \hat{ }180 \hat{ }190
  Y I R P G D A V V I L H V S P T S V L F G A D W G P L P L Q T P P
TTACATCCGTCCCGGAGACGCCGTCGTCATTCTCCACGTTTCTCCAACCTCCGTTCTCTTCGGCGCCGATTGGGGACCTCTCCCTCTCCAAACTCCTCCT
                  \hat{ }210 \hat{ }220 \hat{ }230 \hat{ }240 \hat{ }250 \hat{ }260 \hat{ }270 \hat{ }280 \hat{ }290
P P S A A T D P G A Q P K P S Q E D F D A F T S S K V A D L A K P L
CCTCCTTCCGCCGCTACCGATCCCGGAGCTCAGCCGAAGCCTAGTCAGGAAGATTTCGATGCGTTTACTTCTTCCAAAGTAGCGGATCTAGCGAAGCCGT
                  \hat{ }310 \hat{ }320 \hat{ }330 \hat{ }340 \hat{ }350 \hat{ }360 \hat{ }370 \hat{ }380 \hat{ }390
    K E A G F P H K I H I V K D H D M R E R L C L E T E R L N L S A V
TGAAGGAAGCTGGGTTTCCTCATAAGATCCATATAGTGAAAGATCACGATATGAGAGAGAGGCTTTGCTTAGAGACTGAAAGGCTTAATCTAAGCGCCGT
                  \hat{ }410 \hat{ }420 \hat{ }430 \hat{ }440 \hat{ }450 \hat{ }460 \hat{ }470 \hat{ }480 \hat{ }490
  I M G S R G F G A E K R G S D G K L G S V S D Y C V H H C V C P V
GATAATGGGAAGCAGAGGATTTGGTGCTGAGAAGAGAGGAAGTGATGGCAAGCTTGGCTCTGTTAGTGATTATTGTGTTCACCATTGTGTTTGTCCTGTT
                  \hat{ }510 \hat{ }520 \hat{ }530 \hat{ }540 \hat{ }550 \hat{ }560 \hat{ }570 \hat{ }580 \hat{ }590
V V V R Y P D D R D G P A P P G N V G A T R E A I V T V K S R R D D
GTTGTTGTTAGATATCCTGATGATCGTGATGGTCCTGCTCCTCCTGGGAATGTTGGAGCTACCAGGGAAGCTATTGTCACTGTTAAATCACGTAGGGATG
                  \hat{ }610 \hat{ }620 \hat{ }630 \hat{ }640 \hat{ }650 \hat{ }660 \hat{ }670 \hat{ }680 \hat{ }690
    D D D D D H D H E A K I A A A A S D H H E H I K D E
ATGACGATGATGATGATGAAGATCATGAGGCTAAGATTGCTGCTGCTGCTTCCGATCATCATGAACACATCAAAGATGAGTAAAGTGCCTTAGTATCTCA
                  \hat{ }710 \hat{ }720 \hat{ }730 \hat{ }740 \hat{ }750 \hat{ }760 \hat{ }770 \hat{ }780 \hat{ }790
TCAGGTTCACCCTCTTCACAGTCAGGTACAACGATGGATGAAACAAAACGGATTTGTGCAGAGGAGTGTGTTTGTGTGTTTGTTTATTGCTATCTTTTGC
                  \hat{ }810 \hat{ }820 \hat{ }830 \hat{ }840 \hat{ }850 \hat{ }860 \hat{ }870 \hat{ }880 \hat{ }890
AATATTGAAAAGTCTCTGGAATCTCCTCTGGATTATTTATTCCCAGTCCTTGTTCTTCTTATAATTCTCCATGTATGATTTTCACATTTAAACAATGCCC
                  \hat{ }910 \hat{ }920 \hat{ }930 \hat{ }940 \hat{ }950 \hat{ }960 \hat{ }970 \hat{ }980 \hat{ }990
TTTTCCATGTATGACTTTCACATTTAAACAATACCCTTTTGGGGTTATGACCTTAATCTTCAATCTATTTGATATCAAAAAAAAAAAAAAAAA
                  \hat{ }1010 \hat{ }1020 \hat{ }1030 \hat{ }1040 \hat{ }1050 \hat{ }1060 \hat{ }1070 \hat{ }1080 \hat{ }10
```

1. A method for assaying for the presence or activity of a MAP kinase in a sample, the method comprising:
(a) contacting the sample under conditions necessary for a phosphorylation reaction with a peptide substrate comprising the following target motif:
HP(x)SPR
wherein: (x) represents any amino acid;
(b) assessing any resulting phosphorylation of the peptide substrate; and
(c) correlating the result from step (b) with the presence or activity of MAP kinase in the sample.

2-14. (canceled)

15. A MAP kinase peptide substrate which has the structure:
R₁--(y)ₙ₁--HP(x)SPR--(y)ₙ₂--R₂,
wherein:
HP(x)SPR is a kinase target motif;
R₁ is an organic molecule or an amino terminus;
R₂ is an organic molecule, which is optionally a carboxy terminus or a derivative thereof;
(y)n represents a contiguous sequence of amino acids;
n1 is 0 or an integer of 1 to 500; and
n2 is 0 or an integer of 1 to 500;
and wherein the peptide is selected from the group consisting of:
(a) sequences being overall 70, 80, 90, 95, or 99% identical to all or part of SEQ ID NO: 2; and
(b) sequences encoded by a sequence shown in Table 1.

16. A peptide substrate as claimed in claim 15 wherein (y)ₙ represents a contiguous sequence of amino acids found in SEQ ID No 2, wherein n1 is an integer of 1 to 16, and wherein n2 is an integer of 2 to 238.

17. A peptide substrate as claimed in claim 15 wherein the target motif is present within a contiguous sequence of at least 10, 20, 30, or 40 amino acids depicted in one of SEQ ID NO: 2 or 3 or encoded by a sequence shown in Table 1 or Table 2.

18. A peptide substrate as claimed in claim 15 wherein the peptide is depicted in one of SEQ ID NO: 2 or 3 or a encoded by a sequence shown in Table 1 or Table 2 or a part thereof.

19. A peptide substrate as claimed in claim 15 wherein the peptide comprises a plurality of said target motifs.

20-21. (canceled)

22. A recombinant nucleic acid construct comprising a nucleotide sequence encoding a MAP kinase peptide substrate as of claim 15.

23. (canceled)

24. A nucleic acid construct as claimed in claim 22 which is an expression vector wherein the nucleotide sequence encoding the substrate is operably linked to a promoter for transcription in a host cell.

25. A host cell transformed with a nucleic acid construct as claimed in claim 22.

26. (canceled)

27. A method of transforming a host cell which method comprises introduction of a nucleic acid construct as claimed in claim 22 into a host cell and causing or allowing recombination between the nucleic acid and the cell genome to introduce the nucleotide sequence encoding the MAP kinase peptide substrate into the genome.

28. A process for producing a MAP kinase peptide substrate which process comprises expressing a nucleic acid as claimed in claim 22 in a host cell.

29. (canceled)

30. A method for screening for a compound that modulates MAP kinase-mediated phosphorylation, comprising detecting whether there is a change in the level of MAP kinase-mediated phosphorylation of a peptide substrate of claim 15 in the presence of a candidate compound, wherein an increase in the level of phosphorylation indicates that the compound agonizes MAP kinase-mediated phosphorylation, and a decrease in the level of phosphorylation indicates that the compound antagonizes MAP kinase-mediated phosphorylation.

31-32. (canceled)

33. A composition comprising a MAP kinase peptide substrate of claim 15.

34. (canceled)

35. A kit comprising a peptide substrate of claim 15, and at least one component selected from the group consisting of:
(a) labeled ATP;
(b) printed instructions for performing an assay as described herein;
(c) a MAP kinase;
(d) a protein phosphatase; and
(e) first and second antibodies, which antibodies discriminate between a phosphorylated and a non-phosphorylated peptide substrate.

36. A recombinant nucleic acid vector which comprises a nucleotide sequence encoding a MAP kinase substrate which is capable of altering the salt and\or drought tolerance phenotype of a plant into which the vector is introduced and expressed.

37. A nucleic acid vector as claimed in claim 36 wherein the nucleotide sequence encodes:
(i) an AtPhos34 MAP kinase substrate (SEQ ID NO: 3), or
(ii) a variant thereof having salt- and\or drought-tolerance modifying activity.

38. A nucleic acid vector as claimed in claim 36 wherein the nucleotide sequence is selected from the group consisting of:
(a) sequences shown in Table 1;
(b) SEQ. ID NO: 7 and SEQ ID NO: 6; and
(c) sequences that are degenerately equivalent to a sequence of (a) or (b).

39. A nucleic acid vector as claimed in claim 36 wherein the nucleotide sequence encodes a derivative of the AtPhos34 MAP kinase substrate (SEQ ID NO: 3) by way of addition, insertion, deletion or substitution of one or more amino acids.

40. A nucleic acid vector as claimed in claim 36 wherein the nucleotide sequence is a homologue of SEQ ID NO: 7.

41. A nucleic acid vector as claimed in claim 38 wherein the nucleotide sequence is at least 70, 75, 80, 85, 90, 95, or 99% identical to SEQ ID NO: 7.

42-43. (canceled)

44. A nucleic acid vector as claimed in claim 36 wherein the nucleotide sequence is operably linked to a promoter for transcription in a host cell.

45. A method which comprises the step of introducing the nucleic acid vector of claim 36 into a host cell, and optionally causing or allowing recombination between the nucleic acid and the host cell genome such as to transform the host cell.

46. A host cell containing or transformed with a heterologous nucleic acid of claim 36.

47. A method for producing a transgenic plant, which method comprises the steps of:
(a) performing a method as claimed in claim 45 wherein the host cell is a plant cell, and
(b) regenerating a plant from the transformed plant cell.

48. A transgenic plant produced by the method of claim 47, or a clone, or selfed or hybrid progeny or other descendant of said transgenic plant, or a part or propagule of said transgenic plant.

49. A plant as claimed in claim 48 which is selected from the list consisting of:
tomato, nicotiana, and brassica.

50-51. (canceled)

52. A method for influencing the salt and\or drought tolerance phenotype of a plant, which method comprises the step of causing or allowing expression of a heterologous nucleic acid vector as claimed in claim 36 within cells of the plant, following an earlier step of introducing the nucleic acid vector into a cell of the plant or an ancestor thereof.

53. (canceled)

54. A siRNA duplex containing between 20 and 25 bps of the AtPhos34 sequence (SEQ ID NO: 6).

55-57. (canceled)


SEQUENCE LISTING (12 sequences)

SEQ ID NO: 1 - protein, 169 aa, Bos taurus
AAQKRPSQRSKYLASASTMDHARHGFLPRHRDTGILDSLGRFFGSDRGAPKRGSGKDGHHAARTTHYGSLPQKAQGHRPQDENPVVHFFKNIVTPRTPPPSQGKGRGLSLSRFSWGAEGQKPGFGYGGRASDYKSAHKGLKGHDAQGTLSKIFKLGGRDSRSGSPMARR

SEQ ID NO: 2 - protein, 242 aa, Arabidopsis thaliana
MNPADSDHPQLPNIKIHHPPSPRHSHHHHSSSTPSSAATPTPTAGARRKIGVAVDLSEESSFAVRWAVDHYIRPGDAVVLLHVSPTSVLFGADWGPLPLKTQIEDPNAQPQPSQEDFDAFTSTKVADLAKPLKELGFPYKIHIVKDHDMRERLCLEIERLGLSAVIMGSRGFGAEKKRGSDGKLGSVSDYCVHHCVCPVVVVRYPDDRDGPVPIVTVKSGGDDDGDVVAASASAHHEHIKDE

SEQ ID NO: 3 - protein, 260 aa, Arabidopsis thaliana
MNPDSDYPHLPNIKIHHPSSPRHSHHHSSSTPSAATPTPTAGARRKIGVAVDLSEESAFAVRWAVDHYIRPGDAVVILHVSPTSVLFGADWGPLPLQTPPPPSAATDPGAQPKPSQEDFDAFTSSKVADLAKPLKEAGFPHKIHIVKDHDMRERLCLETERLNLSAVIMGSRGFGAEKRGSDGKLGSVSDYCVHHCVCPVVVVRYPDDRDGPAPPGNVGATREAIVTVKSRRDDDDDDDEDHEAKIAAAASDHHEHIKDE

SEQ ID NO: 4 - DNA, 1026 bp, Arabidopsis thaliana
gaacaaaaacaacatctgaaaaaaatcatgaatccagcagattccgatcatccacagcttccaaacatcaagatccatcaccctccatctccacgtcactctcaccatcaccactcctcatctactccctcctccgccgcaactccaacaccaaccgccggagctcgtcgtaaaatcggagtcgcagttgatctctccgaagaaagctctttcgccgttcgttgggctgtagatcactacatccgtcccggagacgccgttgttcttctccacgtttctccaacctccgtcctcttcggtgccgattggggacctctccctctgaaaactcaaattgaagatccaaacgctcaacctcaacctagtcaagaggattttgatgcttttacttcaacaaaagttgcggatctagctaaaccgttgaaggagttagggtttccttataagatccatatagtgaaagatcatgatatgagagagagattatgtcttgagattgagaggcttggtcttagtgctgtgattatgggaagtagaggttttggtgctgagaaaaaaagaggaagtgatggcaagcttggctctgttagtgattactgtgttcatcactgtgtttgtcctgttgttgtggttagatatcctgatgatcgtgatggacctgtgcctattgttactgtcaagtctggtggagatgatgatggagatgttgttgctgcttctgcttctgctcatcatgaacacatcaaagatgagtgaggatctaagtaatatatcaccctcttctcttctaggtacacaaagcaagtaggggtgttagcggaatgttcagaggagtgttgaatgtgtgttctttctttagctatgacaatgttgatgaatttctttgggttatgtaatatgaatgagtgttgagtggtttctgcattaaacatgaaggagttgtatgattaatgaaccccttttaggaagaagaaggattatacgtttctttaataaagactggtattttagaaaaaaaaaaaaaaa

SEQ ID NO: 5 - DNA, 999 bp, Arabidopsis thaliana
  CDS (1)..(729)
atgaatccagcagattccgatcatccacagcttccaaacatcaagatccatcaccctccatctccacgtcactctcaccatcaccactcctcatctactccctcctccgccgcaactccaacaccaaccgccggagctcgtcgtaaaatcggagtcgcagttgatctctccgaagaaagctctttcgccgttcgttgggctgtagatcactacatccgtcccggagacgccgttgttcttctccacgtttctccaacctccgtcctcttcggtgccgattggggacctctccctctgaaaactcaaattgaagatccaaacgctcaacctcaacctagtcaagaggattttgatgcttttacttcaacaaaagttgcggatctagctaaaccgttgaaggagttagggtttccttataagatccatatagtgaaagatcatgatatgagagagagattatgtcttgagattgagaggcttggtcttagtgctgtgattatgggaagtagaggttttggtgctgagaaaaaaagaggaagtgatggcaagcttggctctgttagtgattactgtgttcatcactgtgtttgtcctgttgttgtggttagatatcctgatgatcgtgatggacctgtgcctattgttactgtcaagtctggtggagatgatgatggagatgttgttgctgcttctgcttctgctcatcatgaacacatcaaagatgagtgaggatctaagtaatatatcaccctcttctcttctaggtacacaaagcaagtaggggtgttagcggaatgttcagaggagtgttgaatgtgtgttctttctttagctatgacaatgttgatgaatttctttgggttatgtaatatgaatgagtgttgagtggtttctgcattaaacatgaaggagttgtatgattaatgaaccccttttaggaagaagaaggattatacgtttctttaataaagactggtattttagaaaaaaaaaaaaaaa

SEQ ID NO: 6 - DNA, 1138 bp, Arabidopsis thaliana
aaattgttgttttatggtgcgtctgcggtacgggtgcggaacgatcatgaatccagattccgattatcctcatctccctaacatcaagatccaccatccttcatctcctcgtcactctcaccaccactcttcctccactccttccgccgctactcctaccccaaccgccggtgctcgccgtaagatcggagtcgccgttgacctttccgaagaaagcgctttcgctgttcgctgggctgtcgatcattacatccgtcccggagacgccgtcgtcattctccacgtttctccaacctccgttctcttcggcgccgattggggacctctccctctccaaactcctcctcctccttccgccgctaccgatcccggagctcagccgaagcctagtcaggaagatttcgatgcgtttacttcttccaaagtagcggatctagcgaagccgttgaaggaagctgggtttcctcataagatccatatagtgaaagatcacgatatgagagagaggctttgcttagagactgaaaggcttaatctaagcgccgtgataatgggaagcagaggatttggtgctgagaagagaggaagtgatggcaagcttggctctgttagtgattattgtgttcaccattgtgtttgtcctgttgttgttgttagatatcctgatgatcgtgatggtcctgctcctcctgggaatgttggagctaccagggaagctattgtcactgttaaatcacgtagggatgatgacgatgatgatgatgaagatcatgaggctaagattgctgctgctgcttccgatcatcatgaacacatcaaagatgagtaaagtgccttagtatctcatcaggttcaccctcttcacagtcaggtacaacgatggatgaaacaaaacggatttgtgcagaggagtgtgtttgtgtgtttgtttattgctatcttttgcaatattgaaaagtctctggaatctcctctggattatttattcccagtccttgttcttcttataattctccatgtatgattttcacatttaaacaatgcccttttccatgtatgactttcacatttaaacaatacccttttggggttatgaccttaatcttcaatctaatttgatatcaaaaaaaaaaaaaaa

SEQ ID NO: 7 - DNA, 1092 bp, Arabidopsis thaliana
  CDS (1)..(783)
atgaatccagattccgattatcctcatctccctaacatcaagatccaccatccttcatctcctcgtcactctcaccaccactcttcctccactccttccgccgctactcctaccccaaccgccggtgctcgccgtaagatcggagtcgccgttgacctttccgaagaaagcgctttcgctgttcgctgggctgtcgatcattacatccgtcccggagacgccgtcgtcattctccacgtttctccaacctccgttctcttcggcgccgattggggacctctccctctccaaactcctcctcctccttccgccgctaccgatcccggagctcagccgaagcctagtcaggaagatttcgatgcgtttacttcttccaaagtagcggatctagcgaagccgttgaaggaagctgggtttcctcataagatccatatagtgaaagatcacgatatgagagagaggctttgcttagagactgaaaggcttaatctaagcgccgtgataatgggaagcagaggatttggtgctgagaagagaggaagtgatggcaagcttggctctgttagtgattattgtgttcaccattgtgtttgtcctgttgttgttgttagatatcctgatgatcgtgatggtcctgctcctcctgggaatgttggagctaccagggaagctattgtcactgttaaatcacgtagggatgatgacgatgatgatgatgaagatcatgaggctaagattgctgctgctgcttccgatcatcatgaacacatcaaagatgagtaaagtgccttagtatctcatcaggttcaccctcttcacagtcaggtacaacgatggatgaaacaaaacggatttgtgcagaggagtgtgtttgtgtgtttgtttattgctatcttttgcaatattgaaaagtctctggaatctcctctggattatttattcccagtccttgttcttcttataattctccatgtatgattttcacatttaaacaatgcccttttccatgtatgactttcacatttaaacaatacccttttggggttatgaccttaatcttcaatctaatttgatatcaaaaaaaaaaaaaaa

SEQ ID NO: 8 - protein, 6 aa, Artificial sequence
  Target motif included in substrate for MAP kinases
HPXSPR

SEQ ID NO: 9 - protein, 6 aa, Arabidopsis thaliana
HPPSPR

SEQ ID NO: 10 - protein, 6 aa, Arabidopsis thaliana
HPSSPR

SEQ ID NO: 11 - protein, 5 aa, Arabidopsis thaliana
PPSPR

SEQ ID NO: 12 - protein, 5 aa, Arabidopsis thaliana
PSSPR